(19) United States
(12) Patent Application Publication
(10) Pub. No.: US 2011/0208059 A1
(43) Pub. Date: Aug. 25, 2011

(54) Ultrasound probe

(75) Inventor: Marino Cerofolini, Subbiano (AR) (IT)

(73) Assignee: ESAOTE S.p.A., Milano (IT)

(21) Appl. No.: 13/031,283

(22) Filed: Feb. 21, 2011

(30) Foreign Application Priority Data
Feb. 23, 2010 (IT) GE2010A000018

(51) Int. Cl. A61B 8/14

(52) U.S. Cl. 600/447

(57) ABSTRACT

An ultrasound probe for diagnostic images includes a set or array of electroacoustic transducers generating an ultrasonic beam and defining at least one scan plane or a predetermined scan volume, and one or more layers matching acoustic impedance characteristics of the transducers with acoustic impedance characteristics of tissues under examiantion. The layers overlap the transducers, on the face emitting/receiving acoustic pulses. Acoustic properties of the material or materials constituting the one or more layers, the geometric shape of the layers, and/or the structure of the transducers cause the ultrasonic beam to be apodized and the profile of the ultrasonic beam in a plane perpendicular to the scan plane and parallel to the ultrasonic beam to be highly homogeneous. The emitted pulse has a greater intensity uniformity in the part of the ultrasonic beam closer to the scan plane and a predetermined lower intensity in side lobes of the ultrasonic beam.

### FIELD OF THE INVENTION

The present invention relates to an ultrasound probe for diagnostic images that includes a set or array of electroacoustic transducers configured to generate an ultrasonic beam to be introduced into a body under examination, the electroacoustic transducers being arranged such to define one or more scan planes.

### BACKGROUND OF THE INVENTION

A type of ultrasound probes, generally called array probes, may be, for example, linear or convex phased array probes, and are widely used for diagnostic purposes in several technical fields and above all in the medical field.

The major advantage of such probes is having a predetermined amount of electroacoustic transducers arranged side by side only along one line (linear probe) or along two or more lines (two-dimensional probe) such to generate an ultrasonic beam whose space features, called focusing, can be electronically controlled by timing the emission of each individual electroacoustic transducer of the array.

Thus, it is possible to control the ultrasonic beam profile on the scan plane, that is the plane passing substantially through the centre of each electroacoustic transducer of the array, along which the wavefront travels and echoes generated by the body under examination are reflected in order to be detected by the probe.

Therefore, that scan plane corresponds to the image plane.

On the contrary, for a plane perpendicular to the array but transverse to such scan plane, for example perpendicular thereto, the beam profile is hard to be controlled since it substantially corresponds to the wavefront defined by the natural aperture of an individual electroacoustic transducer.

This means that on the plane transverse to the scan plane neither the presence of side lobes in the ultrasonic beam nor the presence of an unshaped acoustic field in the near field can be avoided with electronic controls, and therefore the profile of said ultrasonic beam is not very homogeneous.

While in the scan plane, as already pointed out above, a focusing of the energy at different sites of the scan plane can be defined by controlling phase shifting of acoustic pulses upon emission or reception, in the transverse plane such focusing cannot be electrically controlled since the beam profile corresponds to that of an individual transducer, as various electroacoustic transducers to be controlled with possible emission phase shifting are not provided on that transverse plane.

This substantial non-homogeneity leads to a signal response from reflectors arranged on the transverse plane in areas farther from the scan plane and this causes the reflected signal to be subjected to interferences and degradation.

In order to overcome such drawbacks different solutions have been envisaged, such as the use of several acoustic lenses or the partition of each individual electroacoustic transducer into several electronically controlled sub-elements with a variable aperture and possibly with variable delays for a deep focusing.

This second solution, in particular, provides several sub-elements, arranged according to a direction transverse to the scan plane, which can be electronically controlled in order to carry out a focusing also in the transverse direction.

However, those solutions do not provide satisfactory results, or results important enough to justify the related increase in complexity and manufacturing costs.

### SUMMARY OF THE INVENTION

The present invention aims at overcoming the above mentioned drawbacks of known probes.

Such aim is achieved by providing a probe having the features defined hereinbefore and further having one or more layers provided to match acoustic impedance characteristics of transducers with acoustic impedance characteristics of tissues of the body under examination, such layers overlapping the transducers, on the face emitting and receiving acoustic pulses by the transducers.

According to the invention, the acoustic properties of the material or materials constituting said one or more acoustic impedance matching layers and/or the geometric shape of said acoustic impedance matching layers and/or the material and/or the structure of the transducers are such that the ultrasonic beam emitted from said electroacoustic transducers is apodized, so that the profile of said ultrasonic beam in a transverse plane perpendicular to the scan plane and parallel to the direction of propagation of the ultrasonic beam has a substantial high homogeneity level, the emitted ultrasonic beam having a predetermined higher intensity uniformity in the part of said ultrasonic beam closer to the scan plane and a predetermined lower intensity of side lobes of said ultrasonic beam.

Thus, advantageously, the one or more layers already provided within the probe are used, which match the acoustic impedance of tissues of the body under examination with the acoustic impedance of electroacoustic transducers, and therefore allow the energy to be transferred at the greatest extent between the electroacoustic transducers and tissues of the body under examination, thereby guaranteeing a greater sensitivity and bandwidth, for the ultrasonic beam to be apodized on the transverse plane such to concentrate the acoustic energy in a central area in the vicinity of the scan plane and to increase the difference in the acoustic energy between such central area and the peripheral areas farther from the scan plane.

The use of pre-existing elements, with only some simple structural changes to be applied thereto, provides for a very simple manufacturing process and for containing costs.

In a first embodiment according to the invention, the ultrasound probe is provided with transducers made of a piezoelectric composite material.

Such transducers are known and advantageously have a high mechanical coupling coefficient and low acoustic impedance, and are generally made of a ceramic material and a polymer material, the two materials being firmly joined together according to predetermined ratios and geometries.

For example, the transducers made of composite material can be composed of piezoelectric material with notches filled with resin.

In one embodiment, such transducers have such a specific structure of the composite material where the ceramic/resin ratio is not constant but changes at least from the center of the transducer (line of the scan plane) to the outside thereof

More particularly, in order to achieve the apodization effect the ceramic/resin ratio is not constant but decreases from the centre of the transducer (line of the scan plane) to the outside thereof

According to a second alternative embodiment, electroacoustic transducers are of the so called CMUT type (Capacitive Micromachined Ultrasonic Transducers).

Such transducers are composed of capacitive electrostatic micro cells with variable capacity made of a metalized membrane constituting the first electrode and supported over a heavily doped silicon substrate, upon which a second electrode is secured.

Ultrasounds are generated and received by changing the electrostatic force between the two electrodes.

This type of transducers has some advantages, for example has a wide bandwidth and ease of fabrication and integration with other electronic components.

In this second embodiment, similar to the above description for piezoelectric composite transducers, the emitted ultrasonic beam may be apodized on the transverse plane by changing, for example, the density of electrostatic micro cells starting from the central part of the transducer to the periphery thereof.

More particularly, the apodization effect is achieved, for example, by reducing the density of the electrostatic micro cells starting from the central part of the transducer to the periphery thereof.

In a third embodiment of the invention, the ultrasound probe is provided only with one acoustic impedance matching layer, which has a lower acoustic absorption in the central part closer to the scan plane and a greater acoustic absorption in side parts farther from the scan plane.

This embodiment has the advantage of a very simple construction since only one acoustic impedance matching layer is required, having an absorption which changes depending on the distance from the center.

Such variability in the absorption is obtained by modifying, particularly by doping, a homogeneous material with other materials that have predetermined acoustic properties and that are embedded with a variable distribution with regard to the distance from the center, such that the result of absorbing acoustic energy in the farther areas from the scan plane is achieved.

A fourth embodiment provides for an additional acoustic impedance matching layer overlapping the first one and at least one of such acoustic impedance matching layers has at least a curved surface.

More particularly, the interface surface between the two layers has a concave shape, such that the layer overlapping the electroacoustic transducers is thicker at the peripheral parts of the electroacoustic transducers of the array and it is thinner at the central electroacoustic transducers of the array.

In another embodiment, at least one of the acoustic impedance matching layers is made of a material filled with glass and/or ceramic powder or the like.

This allows obtaining layers made of a doped material, wherein the acoustic absorption can be changed by acting on the amount of powder contained therein and on the particle size thereof.

In a preferred embodiment, the acoustic impedance matching layer adjacent to the electroacoustic transducers is an apodizing layer made of a sound absorbing material.

The layer contacting the body under examination on the contrary is a focusing layer made of non-absorbing material.

The focusing layer is made of a material having a propagation velocity slower than the propagation velocity of the material constituting the apodizing layer, such that the difference in the propagation velocity between said focusing layer and said apodizing layer constitutes an acoustic lens allowing for the ultrasonic beam to be acoustically focused in a predetermined site.

In a variant embodiment, two additional acoustic impedance matching layers are provided between the electroacoustic transducers and the apodizing layer.

Therefore, a multi-layer is obtained further acting for minimizing internal reflections.

Moreover, it is possible to have the two additional acoustic impedance matching layers used as resonator elements, with the advantage of obtaining a wider transmission band.

In fact, the thickness of the transducers, the thickness of the transducer and the first acoustic impedance matching layer, and the thickness of the transducer and the first and the second acoustic impedance matching layer, define three different resonance frequencies because there is a difference in the acoustic impedance of the transducers and of the acoustic impedance matching layers, thus causing reflections at the interfaces.

In the transmission spectrum, when the acoustic energy component relative to the first resonance frequency begins to fade, the increase beging relative to the second resonance frequency and so on.

This can allow obtaining a wider transmission band.

In an embodiment, the two additional acoustic impedance matching layers have the same thickness.

In another embodiment, the two additional acoustic impedance matching layers have different thickness.

In still another embodiment, at least one of the two further acoustic impedance matching layers has the same thickness of the electroacoustic transducers and/or the apodizing layer.

In still another embodiment, the apodizing layer is made of polymer material, preferably resin.

In still annother embodiment, the material constituting the apodizing layer, preferably the resin, is filled with one or more ceramic powders or the like, preferably alumina or glass, with a particle size preferably ranging from 80 to 200 μm.

In still another embodiment, the focusing layer is made of an elastomeric material, preferably silicone or the like.

Annother embodiment provides for the material constituting the focusing layer, preferably silicone as mentioned above or the like, to be filled with one or more fine powders, with a particle size preferably ranging from 1 to 10 μm. Advantageously, this does not generate absorption.

### BRIEF DESCRIPTION OF THE DRAWINGS

These and other characteristics and advantages of the present invention will be more clear from the following description of some embodiments shown in attached drawings, wherein:

FIG. 1 is a front view of a probe according to the present invention;

FIG. 2 is a bottom view of the probe of FIG, 1, taken from the ultrasonic pulse emitting face;

FIG. 3 is a side view of the probe of FIG. 1;

FIG. 4 is a diagram of the distribution of acoustic energy in a transverse plane without apodization;

FIG. 5 is a diagram of the distribution of acoustic energy in a transverse plane with apodization;

FIG. 6 is a view showing the transducer array taken from the acoustic pulse emitting face;

FIGS. 7 and 8 are two embodiments of an individual electroacoustic transducer taken from the acoustic pulse emitting face;

FIG. 9 is a side view of an embodiment of the acoustic impedance matching layer; and

FIGS. 10 and 11 are front views of two additional embodiments of the acoustic impedance matching layers.

### DETAILED DESCRIPTION OF EMBODIMENTS OF THE INVENTION

Detailed descriptions of embodiments of the invention are provided herein. It is to be understood, however, that the present invention may be embodied in various forms. Therefore, the specific details disclosed herein are not to be interpreted as limiting, but rather as a representative basis for teaching one skilled in the art how to employ the present invention in virtually any detailed system, structure, or manner.

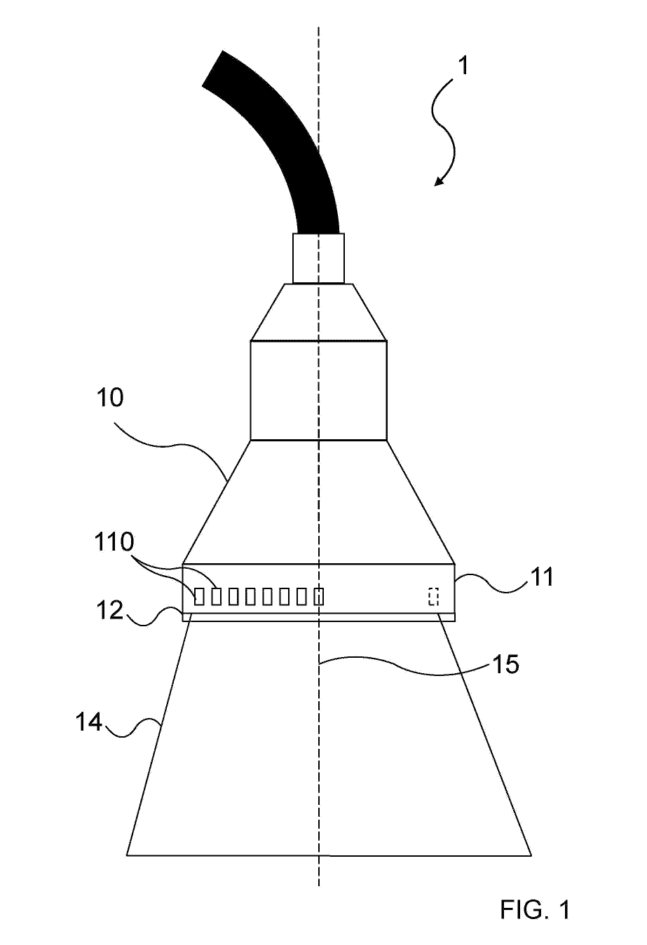

FIG. 1 is a front view, perpendicular to the scan plane, showing a schematic example of a probe according to the present invention.

For simplicity reasons a linear probe is shown; however, it is possible to apply the invention to any type of probe as mentioned hereinbefore.

Construction characteristics of the probe are intentionally schematic since they are not the subject matter of the present invention.

The illustrated embodiment relates to an ultrasound probe 1 for diagnostic images that includes a body 10, and a set or array 11 of electroacoustic transducers 110 for generating an ultrasonic beam to be introduced into the body under examination.

The electroacoustic transducers are arranged along a line such to define a scan plane 14.

The probe also includes one or more layers 12 for matching acoustic impedance characteristics of transducers 110 with acoustic impedance characteristics of tissues of the body under examination, t layers overlapping the transducers 110, on the face emitting and receiving acoustic pulses by transducers 110.

The acoustic properties of the material or materials constituting the one or more acoustic impedance matching layers 12 and/or the geometric shape of the acoustic impedance matching layers 12 and/or the material and/or the structure of the transducers 110 are such that the ultrasonic beam emitted from the electroacoustic transducers 110 is focused and apodized, so that the profile of said ultrasonic beam in a plane 15 transverse to, particularly perpendicular to the scan plane 14 and parallel to the direction of propagation of the ultrasonic beam has a high homogeneity level, the emitted pulse having a higher intensity uniformity in the part closer to the scan plane and a lower intensity of side lobes.

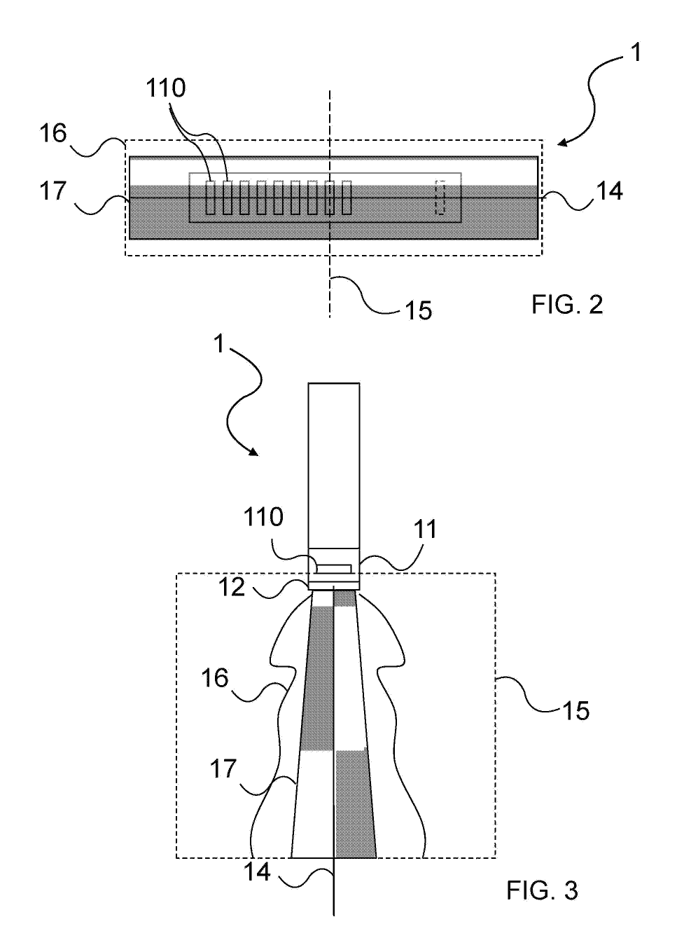

This can be clearly seen in FIGS. 2 and 3, in which a bottom view, which is taken from the ultrasonic pulse emitting face, and a side view of said probe 1 are shown respectively.

The scan plane 14 is the plane substantially passing through the central part of each electroacoustic transducer 110 and corresponds to the image plane.

Actually, the ultrasonic beam will also have a certain aperture in the transverse plane, that is the region with the highest intensity will become wider as the sound pulse travels away from the electroacoustic transducers.

This is schematically shown in FIG. 2 by a rectangle that approximately shows the aperture section of the unapodized ultrasonic beam 16 along a plane perpendicular to the direction of propagation of the ultrasonic pulse at a certain distance from the electroacoustic transducers 110.

FIG. 3 clearly shows said ultrasonic beam 16, and the non-homogeneous profile and the aperture of said beam with respect to the transverse plane 15 are clearly shown.

The beam length on the transverse plane 15 leads to a signal response from reflectors arranged on the transverse plane 15 in areas farther from the scan plane 14, and this leads to interferences and degradation of the reflected signal and to the generation of artifacts with such non uniform beam profile.

According to the present invention, by acting on the geometric shape and on materials constituting the acoustic impedance matching layers and/or the electroacoustic transducers it is possible to obtain an apodization allowing the ultrasonic beam profile to be changed, generating a new apodized ultrasonic beam 17, having a uniformity greater than that of the unapodized ultrasonic beam 16.

Thus the acoustic energy is uniformly distributed from the scan plane to the outside and thus reflection effects generated by structures farther from the scan plane 14 are limited.

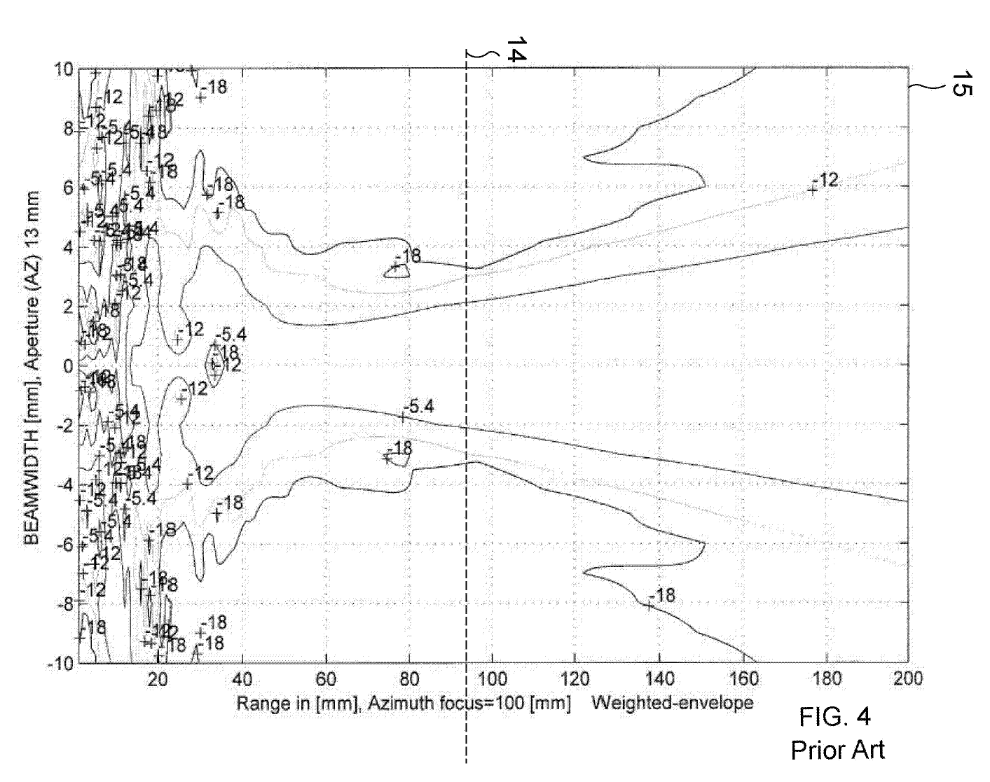

FIG. 4 shows a diagram of the acoustic energy distribution according to the prior art and without apodization in the transverse plane 15. The abscissa represents the distance from the electroacoustic transducers in the direction of the propagation sense of pulses in millimeters, while the ordinate represents the distance from the scan plane 14 in millimeters.

As it can be clearly seen, the acoustic field is not shaped in the near field, having a high non-homogeneity which adversely affects the quality of the reconstructed image, above all in the area close to transducers.

Past the near field, it can be easily seen that the ultrasonic beam has an enlarged and very irregular profile and such non-homogeneities lead to the above mentioned drawbacks.

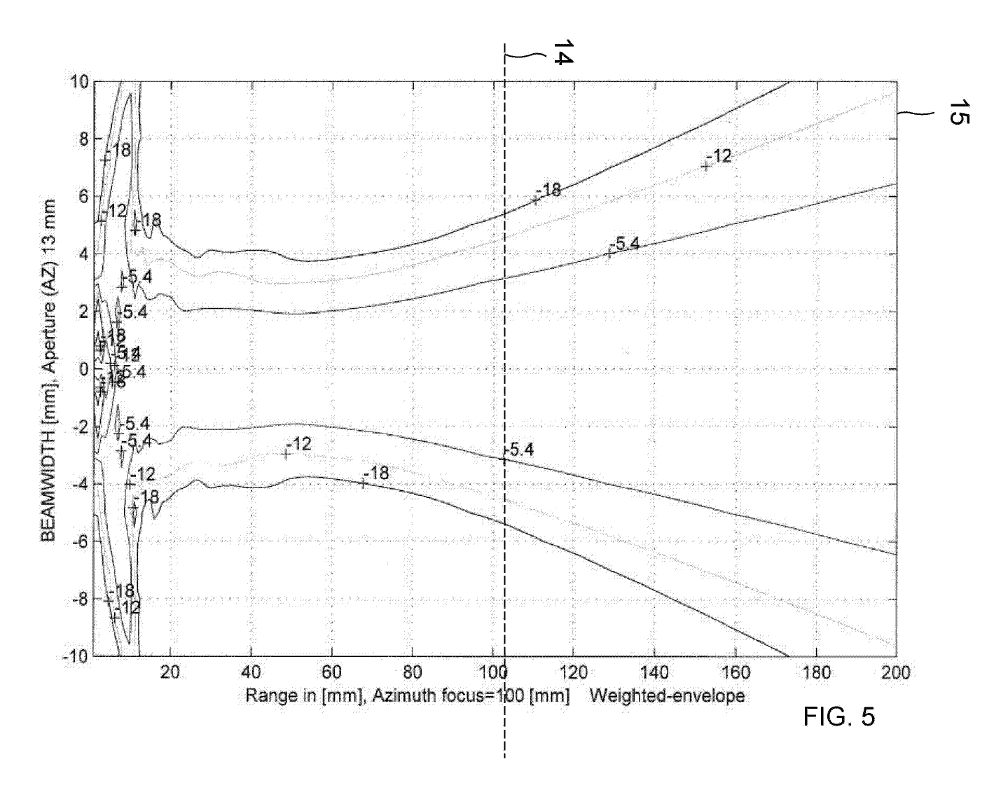

FIG. 5 shows a diagram corresponding to that of FIG. 4, showing the ultrasonic beam profile on the transverse plane 15 generated from an ultrasound probe 1 according to the present invention.

It can be clearly noted that in the near field the state of the energy distribution is much more homogeneous than the corresponding one in FIG. 4, and the same may be said also for the ultrasonic beam profile.

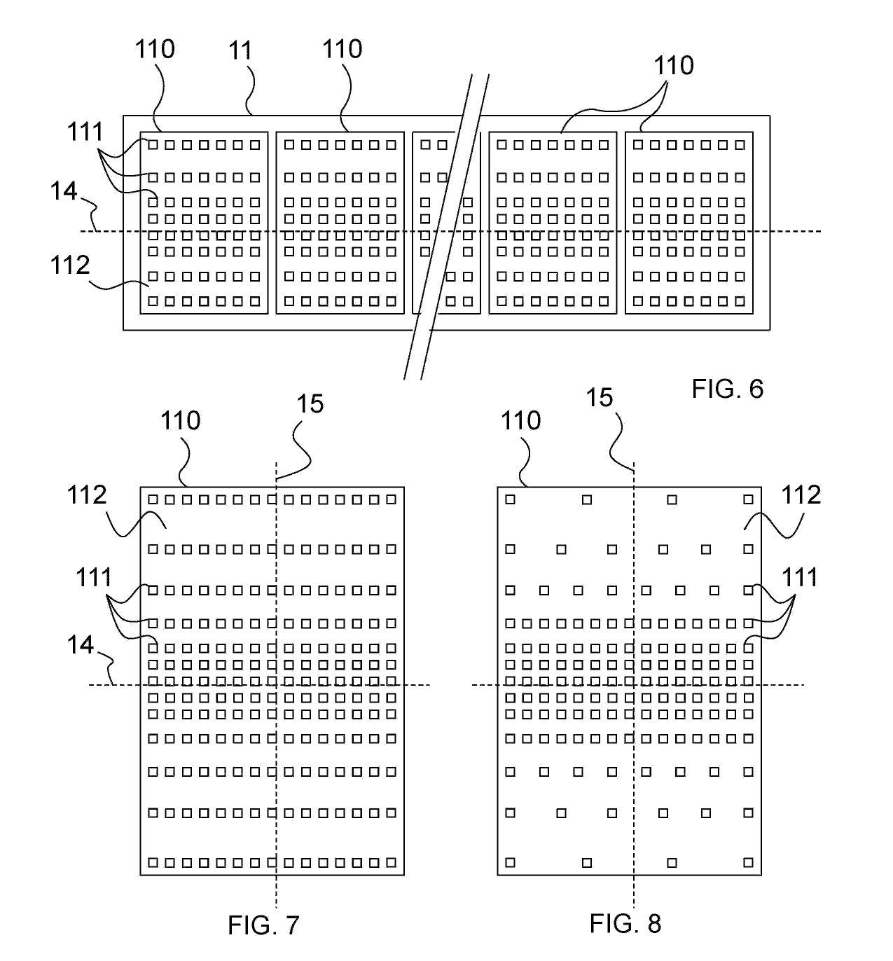

FIG. 6 is a view of the transducer array 11 taken from the acoustic pulse emitting face, wherein transducers 110 are adjacent to one another such to define a scan plane 14 substantially passing through the central part of each transducer.

According to the illustrated embodiment, the transducers are of the ceramic piezoelectric composite type, and comprise ceramic piezoelectric elements 111 buried into a polymer material matrix 112.

Ceramic piezoelectric elements 111 are arranged such to be highly concentrated at the central area of the transducer 110, that is in the vicinity of the scan plane 14, and such to be more spaced apart in the side areas farther from the scan plane 14, such that acoustic pulses are emitted with a higher intensity in the central area of the transducer 110.

In a further embodiment, the transducer is composed of piezoelectric material with notches filled with resin, having such a specific structure that a ceramic/resin ratio decreases as the distance from the scan plane 14 increases.

FIGS. 7 and 8 show two embodiments of an individual electroacoustic transducer taken from the acoustic pulse emitting face, according to the embodiment of FIG. 6. In particular, in FIG. 7 the space density of piezoelectric elements 111 decreases in the direction perpendicular to the scan plane 14 as the distance from the scan plane 14 increases but the piezoelectric elements 111 maintain the same distance from one another in the direction parallel to said scan plane, while in FIG. 8 the space density of the piezoelectric elements 111 decreases as the distance from the scan plane increases 14 both in the direction perpendicular to the scan plane 14 and in the direction parallel to that plane.

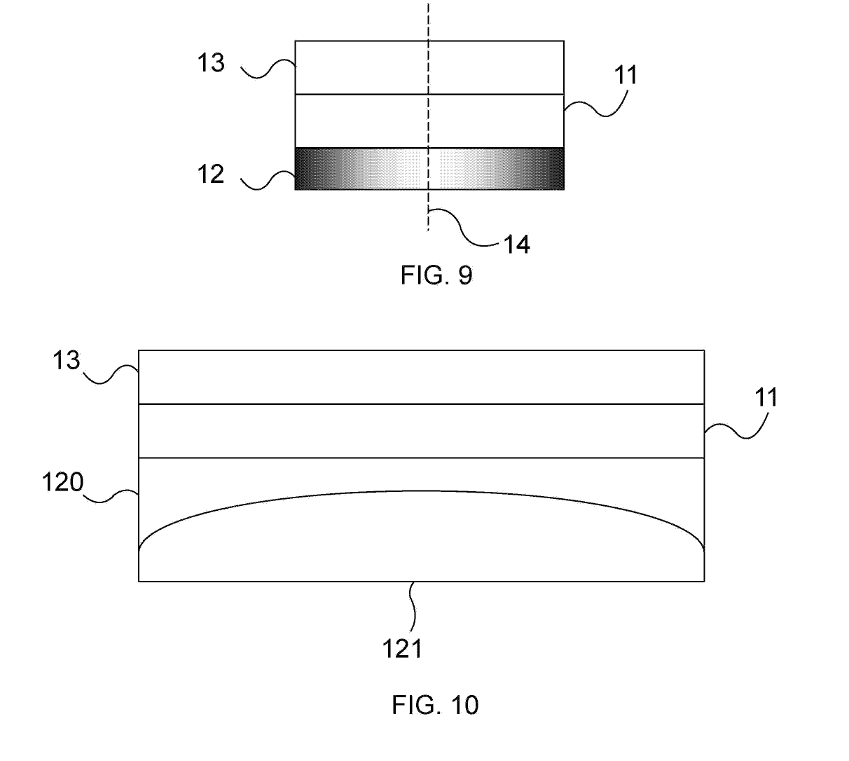

FIG. 9 shows a side view of a first embodiment of the acoustic impedance matching layer according to the invention.

In this embodiment, the ultrasound probe is provided only with one acoustic impedance matching layer 12, which is an apodizing layer 120, and has a lower sound absorption in the central portion closer to the scan plane 14 and a greater sound absorption in the side areas farther from the scan plane 14.

The electroacoustic transducer array 11 on the pulse emitting/receiving face is overlapped by the apodizing layer 120 with a variable absorption depending on the distance from the center, while on the opposite face it has a high absorption backing layer 13 for damping pulses emitted in that direction.

A second embodiment, shown in FIG. 10, provides for a further acoustic impedance matching layer overlapping the apodizing layer 120. The interface surface between the two layers has a concave shape, such that the apodizing layer 120 overlapping the electroacoustic transducer array 11 is thicker at the peripheral opposite parts of the electroacoustic transducers of said array 11 with respect to the scan plane 14 and is thinner at the central part of the electroacoustic transducers of said array 11.

In a further embodiment, at least one of the acoustic impedance matching layers is made of a material filled with glass and/or ceramic powder or the like.

In a preferred embodiment the apodizing layer 120 is made of a sound absorbing material.

On the contrary, the layer in contact with the body under examination is a focusing layer 121 and it is made of a non absorbing material.

The focusing layer 121 is made of a material having a propagation velocity slower than the propagation velocity of the material constituting the apodizing layer 120, such that the difference in the propagation velocity between the focusing layer 121 and the apodizing layer 120 makes an acoustic lens, in order to allow the ultrasonic beam to be acoustically focused in a predetermined site.

In a further embodiment, the apodizing layer 120 is made of polymer material, preferably resin.

In a further embodiment, the material constituting the apodizing layer 120, preferably said resin, is filled with one or more ceramic powders or the like, preferably alumina or glass, with a particle size preferably ranging from 80 to 200 μm.

In a further embodiment, the focusing layer 121 is made of an elastomeric material, preferably silicone or the like.

A further embodiment provides for the material constituting the focusing layer 121, preferably silicone or the like, to be filled with fine powders, with a particle size preferably ranging from 1 to 10 μm.

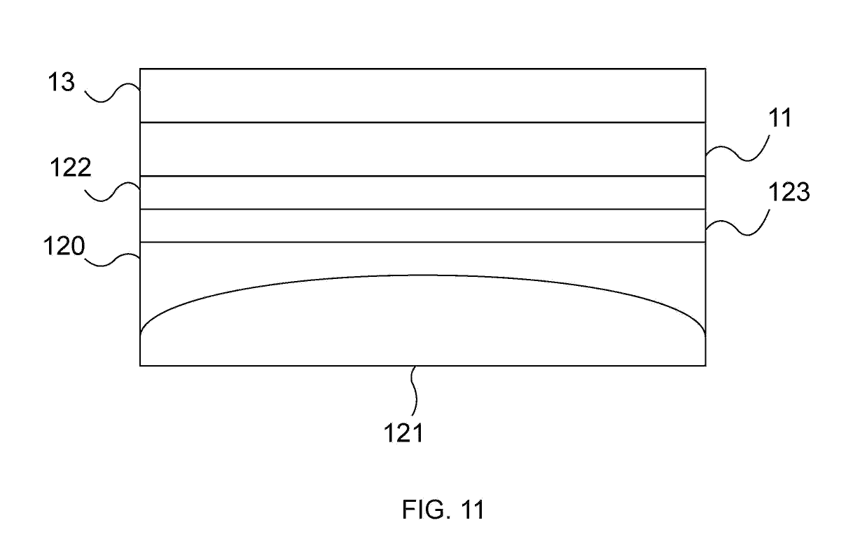

FIG. 11 shows a front view of a further embodiment of the acoustic impedance matching layers, in which two additional acoustic impedance matching layers 122 and 123 are provided between the electroacoustic transducer array 11 and the apodizing layer 120.

In a preferred embodiment, the electroacoustic transducers have an acoustic impedance of about 25 rayls, the apodizing layer 120 has an acoustic impedance of about 3 rayls, the focusing layer has an acoustic impedance substantially equal to that of tissues of the body under examination, and the additional acoustic impedance matching layers 122 and 123 have an acoustic impedance of about 12 rayls and 6 rayls respectively.

The described embodiments of the combinations of layers are not to be intended as limitative since technical results provided by the invention can be obtained with the layers in any combination providing for the same variability of the sound absorption in the direction transverse to the scan plane and substantially of the width of the electroacoustic transducer array.

Moreover, while the invention has been described in connection with the above described embodiments, it is not intended to limit the scope of the invention to the particular forms set forth, but on the contrary, it is intended to cover such alternatives, modifications, and equivalents as may be included within the scope of the invention. Further, the scope of the present invention fully encompasses other embodiments that may become obvious to those skilled in the art and the scope of the present invention is limited only by the appended claims.

1. An ultrasound probe for diagnostic images comprising:
a set or array of electroacoustic transducers configured to generate an ultrasonic beam to be introduced into a body under examination, the electroacoustic transducers being arranged such to define at least one scan plane or a predetermined scan volume; and
means for setting emission acoustic properties of the probe, such that a profile of the ultrasonic beam in a plane transverse or perpendicular to the scan plane and parallel to a direction of propagation of the ultrasonic beam has a high homogeneity level,
wherein an emitted ultrasonic beam has a predetermined higher intensity uniformity in a part of the ultrasonic beam closer to the scan plane and a predetermined lower intensity of side lobes of the ultrasonic beam.

2. The ultrasound probe for diagnostic images of claim 1, further comprising one or more acoustic impedance matching layers that match acoustic impedance characteristics of the electroacoustic transducers with acoustic impedance characteristics of tissues of the body under examination, the one or more acoustic impedance matching layers being overlapped to the electroacoustic transducers, on a face emitting/receiving acoustic pulses by the transducers and one or more acoustic properties of one or more materials comprised in the one or more acoustic impedance matching layers, or a geometric shape of the one or more acoustic impedance matching layers being such that the ultrasonic beam emitted from the electroacoustic transducers is apodized.

3. The ultrasound probe for diagnostic images of claim 2, wherein there is only one acoustic impedance matching layer.

4. The ultrasound probe for diagnostic images of claim 3, wherein the acoustic impedance matching layer has a lower acoustic absorption in a central part closer to the scan plane and a greater acoustic absorption in side parts farther from the scan plane.

5. The ultrasound probe for diagnostic images of claim 2, wherein the one or more acoustic impedance matching layers are two acoustic impedance matching layers overlapping each other.

6. The ultrasound probe for diagnostic images of claim 2, wherein at least one of the one or more acoustic impedance matching layers has at least a curved surface.

7. The ultrasound probe for diagnostic images of claim 5, wherein an interface surface between the two acoustic impedance matching layers has a concave shape, such that the acoustic impedance matching layer overlapping the electroacoustic transducers is thicker at peripheral opposite parts of the electroacoustic transducers of the array with respect to the scan plane and is thinner at a central part of the electroacoustic transducers of the array.

8. The ultrasound probe for diagnostic images of claim 2, wherein at least one of the one or more acoustic impedance matching layers comprises a material filled with glass or a ceramic powder.

9. The ultrasound probe for diagnostic images of claim 2, wherein the acoustic impedance matching layer adjacent to the electroacoustic transducers is an apodizing layer comprising a sound absorbing material and the acoustic impedance matching layer in contact with the body under examination is a focusing layer comprising non-absorbing material, the material of the focusing layer having a propagation velocity slower than a propagation velocity of the material of the apodizing layer, such that a difference in propagation velocity between the apodizing layer and the focusing layer provides an acoustic lens allowing the ultrasonic beam to be acoustically focused in a predetermined site.

10. The ultrasound probe for diagnostic images of claim 9, wherein the apodizing layer comprises a polymer material.

11. The ultrasound probe for diagnostic images of claim 9, wherein the material comprised in the apodizing layer is filled with a ceramic powder.

12. The ultrasound probe for diagnostic images of claim 9, wherein the focusing layer comprises an elastomeric material.

13. The ultrasound probe for diagnostic images of claim 9, wherein the material comprised in the focusing layer is filled with one or more fine powders.

14. The ultrasound probe for diagnostic images of claim 9, further comprising two additional acoustic impedance matching layers between the electroacoustic transducers and the apodizing layer.

15. The ultrasound probe for diagnostic images of claim 14, wherein the two additional acoustic impedance matching layers operate as resonator elements obtaining a wider transmission band.

16. The ultrasound probe for diagnostic images of claim 14, wherein the two additional acoustic impedance matching layers are of a same thickness.

17. The ultrasound probe for diagnostic images of claim 14, wherein the two additional acoustic impedance matching layers are of different thickness.

18. The ultrasound probe for diagnostic images of claim 14, wherein at least one of the two additional acoustic impedance matching layers have same thickness as one or more of the electroacoustic transducers or the apodizing layer.

19. The ultrasound probe for diagnostic images of claim 1, wherein the electroacoustic transducers have a structure that provides a predetermined higher intensity of acoustic emission in a central area, which is an area closer to the scan plane.

20. The ultrasound probe for diagnostic images of claim 19, wherein the electroacoustic transducers comprise a ceramic piezoelectric composite material and have such a structure that a ceramic/polymer ratio changes in a direction of a median plane of the electroacoustic transducers which is perpendicular to the scan plane and depending on a distance from the median plane.

21. The ultrasound probe for diagnostic images of claim 19, wherein the electroacoustic transducers comprise a ceramic piezoelectric composite material and have such a structure that a ceramic/polymer ratio is greater in a vicinity of the scan plane and it decreases as distance from the scan plane increases.

22. The ultrasound probe for diagnostic images of claim 19, wherein the electroacoustic transducers are of a CMUT type and have such a structure that a density of electrostatic cells changes in a direction of a median plane of the electroacoustic transducers which is perpendicular to the scan plane, and depends on distance the median plane.

23. The ultrasound probe for diagnostic images of claim 19, wherein the electroacoustic transducers are of a CMUT type and have such a structure that a density of electrostatic cells is greater in a vicinity of the scan plane and decreases as distance from the scan plane increases.

24. The ultrasound probe for diagnostic images of claim 19, further comprising one or more acoustic impedance matching layers that match acoustic impedance characteristics of the electroacoustic transducers with acoustic impedance characteristics of tissues of the body under examination, the one or more acoustic impedance matching layers being overlapped to the electroacoustic transducers, on a face emitting/receiving acoustic pulses by the transducers and one or more acoustic properties of one or more materials comprised in the one or more acoustic impedance matching layers, or a geometric shape of the one or more acoustic impedance matching layers being such that the ultrasonic beam emitted from the electroacoustic transducers is apodized.
